(19) United States
(12) Patent Application Publication
(10) Pub. No.: US 2002/0156040 A1
(43) Pub. Date: Oct. 24, 2002

(54) Reduction of antioxidant enzyme levels in tumor cells using antisense oligonucleotides

(76) Inventors: Larry Wayne Oberley, Iowa City, IA (US); Christine J. Weydert, Iowa City, IA (US); Benjamin Barnes Smith, Iowa City, IA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 09/993,333

(22) Filed: Nov. 14, 2001

Related U.S. Application Data
(60) Provisional application No. 60/248,328, filed on Nov. 14, 2000.

(51) Int. Cl. A61K 48/00; C07H 21/04

(52) U.S. Cl. 514/44; 536/23.2

(57) ABSTRACT

The present invention provides an antisense oligonucleotide that specifically binds to an antioxidant enzyme start codon so as to inhibit the level of antioxidant enzymes in a cell. The present invention further provides methods of treating an antioxidant enzyme malfunction disorder in a mammal by reducing antioxidant enzyme levels in a cell with the administration of the antisense oligonucleotide.

### RELATED APPLICATION

This application claims priority under 35 U.S.C. 119(e) from U.S. provisional Application No. 60/248,328 filed Nov. 14, 2000, which application is incorporated herein by reference.

### GOVERNMENT FUNDING

The invention described herein was made with U.S. Government support under Grant Number P01 CA66081 awarded by the National Institutes of Health. The United States Government has certain rights in the invention.

### BACKGROUND OF THE INVENTION

The enzymatic activity of antioxidant proteins differs in cancer cells as compared to their normal tissue counterparts. At present there are no products available that can be injected directly into a tumor that will decrease the level of expression of antioxidant enzyme genes.

Therefore, there is an ongoing need for therapeutic agents and methods to efficiently decrease the level of expression of antioxidant enzyme genes.

### SUMMARY OF THE INVENTION

The present invention provides an oligonucleotide that is an antisense nucleic acid sequence that specifically binds to an antioxidant enzyme BRNA start codon, wherein the sequence is about 18 to 26 nucleotides in length, such as about 20 nucleotides long. The nucleic acid is DNA, and the nucleic acid may be phosphothiolated. The antioxidant enzyme mRNA to which the oligonucleotide binds may be manganese superoxide dismutase, copper and zinc superoxide dismutase, catalase, phospholipid glutathione peroxidase, or cytosolic glutathione peroxidase. The nucleic acid sequence may be 90%, or even 100% identical to the nucleic acid encoding an antioxidant enzyme.

The present invention also provides methods of treating an antioxidant enzyme malfunction disorder in a mammal, such as a human, by reducing antioxidant enzyme levels in a cell by administering a therapeutic agent comprising an oligonucleotide described above. The disorder to be treated may be a tumor, heart disease, arthritis, or neurodegenerative disease. The method may involve the injection of the therapeutic agent into a tumor. The therapeutic agent may contain a delivery vehicle, such as lipofectamine or N-[1-(2,3-dioleoyloxy)propyl]-N,N,N-trimethylammonium methyl sulfate (“DOTAP”).

### BRIEF DESCRIPTION OF THE FIGURES

FIG. 1: Human manganese superoxide dismutase (MnSOD) nucleotide and amino acid sequences. MnSOD antisense ODNs were targeted to the ATG start site and are designated as oligo 1, oligo 2 or oligo 3.

FIG. 2A: Western analysis of MnSOD immunoreactive protein in U118-9 human glioma cells after treatment with antisense oligonucleotides.

FIG. 2B: Gel analysis of MnSOD activity in U118-9 human glioma cells after treatment with antisense oligonucleotides.

FIGS. 3A-3C: MnSOD, Catalase Western and GPx native immunoblot analysis of MCF10A and MCF-7 breast cancer cells.

FIG. 4: Comparison of MnSOD protein expression levels. Lane 1 contained control MCF-7, lane 2 contained Effectene (10 μl/ml) and lane 3 contained antisense MnSOD (1 μM).

FIG. 5: Comparison of the effects of Effectene and antisense MnSOD on MCF10A and MCF-7 cells. Control cells are also shown.

FIG. 6: Graph depicting the effects of Effectene and antisense MnSOD on MCF10A and MCF-7 cells as compared to control cells.

FIG. 7A-B: Comparison of MnSOD protein expression levels in MCF10A and MCF-7 cells. Lane 1 contained control MCF-7, lane 2 contained antisense MnSOD (10 μM), lane 3 contained scrambled MnSOD (10 μM), lane 4 contained mismatch MnSOD (10 μM), and lane 5 contained sense MnSOD (10 μM).

FIG. 8: Graph depicting MnSOD protein expression levels in MCF10A and MCF-7 cells treated with antisense MnSOD (10 μM), scrambled MnSOD (10 μM), mismatch MnSOD (10 μM), and sense MnSOD (10 μM) as compared to controls.

FIG. 9: Chart comparing number of disease free mice at day 316 after treatment with various agents.

FIG. 10A-C: Comparison of human melanoma cells treated with 1 μM antisense MnSOD or 10 μl/ml Effectene. Controls are also shown.

FIG. 11: Graph depicting viability of human melanoma treated with antisense MnSOD (1 μM).

### DETAILED DESCRIPTION OF THE INVENTION

The present inventors have discovered that antisense technology can be used to alter the expression of antioxidant enzymes in a mammal, for example, by administering to the mammal an effective amount of “antisense” oligonucleotides. As used herein, the term “antisense” means a sequence of nucleic acid that is the reverse complement of at least a portion of a RNA or DNA molecule that codes for an antioxidant enzyme. The introduction of antioxidant enzyme antisense nucleic acid into a cell ex vivo or in vivo can result in a molecular genetic-based therapy directed to controlling the expression of antioxidant enzyme. Thus, the introduced nucleic acid may be useful to reduce the expression of antioxidant enzyme in mammals with an antioxidant enzyme malfunction disorder. For example, the administration of antisense antioxidant enzyme sequences may be useful to treat an antioxidant enzyme malfunction disorder.

Development of Antisense Reagents for the Antioxidant Proteins

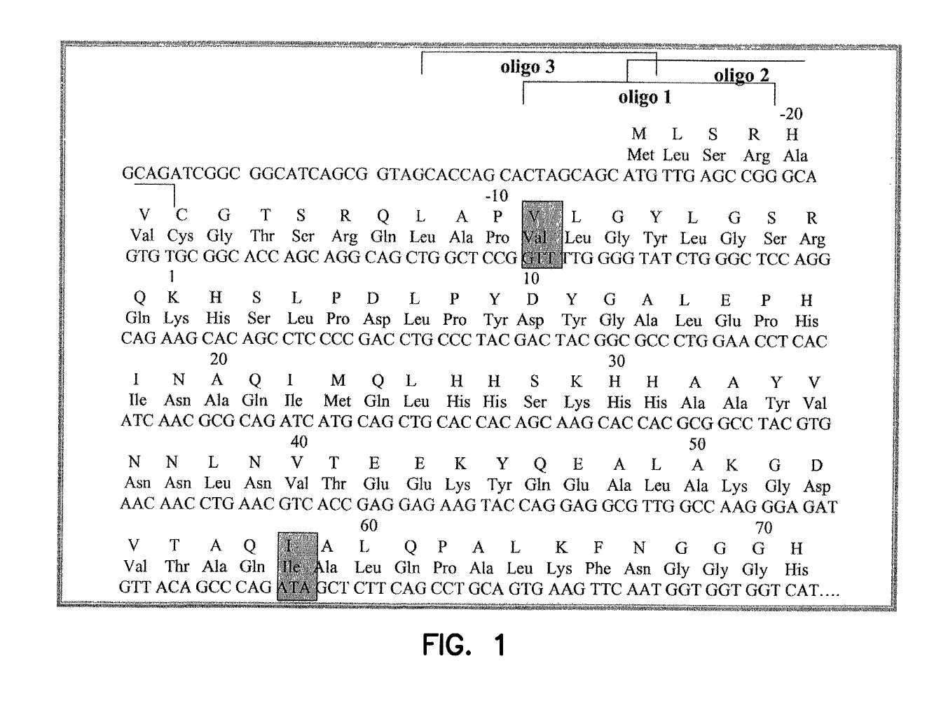

Antisense oligonucleotides (also called “antisense oligos”) are made for the major antioxidant proteins. For example, the present inventors have successfully made antisense oligos for Manganese Superoxide Dismutase (MnSOD) (human MnSOD nucleotide sequence provided in SEQ ID NO: 11, amino acid sequence provided in SEQ ID NO: 12; see FIG. 1) and catalase (CAT) using the following strategy. First, 20-mer sequences were synthesized with the start codon of the coding sequence of the enzyme in question in the center of the oligo. Oligos were then made by shifting the start sequence 5′ and then 3′ from the original sequence. For instance, for MnSOD, the following three oligos were made:

5′ CCG GCT CAA CAT GCT GCT AG (SEQ ID NO: 1)

MnSOD oligo 1, start codon is highlighted

5′ ACA CTG CCC GGC TCA ACA TG (SEQ ID NO: 2)

MnSOD oligo 2, upstream from start codon

5′ CAT GCT GCT AGT GCT GGT GC (SEQ ID NO: 3)

MnSOD oligo 3, downstream from start codon

The oligos can be phosphorothioated on the first six and last six bases for stability. For catalase, the following two constructs were made:

5′ GGA TCC CGG CTG TCA GCC AT (SEQ ID NO: 4)
5′ CAT AGC GTG CGG TTT GCT CT (SEQ ID NO: 5)

The following two phospholipid GPx sequences were made:

5′ GCC GAG GCT CAT CGC GGC GG (SEQ ID NO: 6)
5′ CAA AGG CGG CCG AGG CTC AT (SEQ ID NO: 7)

Development of Antisense Reagents

In most cases overexpression of antioxidant protein protects cancer cells. In the clinic, the opposite effect is desired: one wants to sensitize cancer cell killing. Thus, it is desirable to inhibit antioxidant enzyme levels in order to sensitize to various antitumor modalities.

For this reason, the inventors had the developmental objective of making antisense reagents, in particular antisense oligonucleotides. In a previous collaboration with Dr. Ted Dawson at Johns Hopkins, antisense MnSOD oligos to rat MnSOD were made and it was shown that they inhibit MnSOD protein levels and lowered MnSOD activity. Gonzalez-Zulueta et al., J. of Neuroscience, 18, 2040-2055 (1998). In the rat malignant pheochromocytoma-derived cell line PC12, antisense oligonucleotides almost completely eliminated MnSOD protein levels and catalytic activity, but had no effect on CuZnSOD. Cell viability was not affected by treatment with antisense oligonucleotides alone. Exposure of cells to antisense oligonucleotides sensitized cells dramatically to cell killing induced by nitric oxide or superoxide. Sense or random oligos with equivalent levels of phosphorothioation had no effect on MnSOD protein level, MnSOD activity, or sensitivity to killing. These results show that antisense oligonucleotides can specifically inhibit MnSOD and potentiate cell killing.

It was then tested to see if this concept would work in certain cancer cell types because they have very low levels of MnSOD compared to normal tissue. If this were true, a therapeutic advantage could be set up with the same amount of antisense oligonucleotides inhibiting MnSOD to a near zero level in the cancer tissue while leaving appreciable levels of MnSOD in all normal tissue. This was an entirely new approach that was completely untested prior to the experiments of the present inventors.

Using the methods of the present invention, the antisense oligonucleotides are administered by means of an intratumoral injection. The oligonucleotides are suspended in an appropriate solution, such as water, saline solution or other solution well-known in the art.

The concentration of the oligonucleotides in the therapeutic agent is 1 to 10 μM.

The invention will be further described by reference to the following detailed examples.

### EXAMPLES

### Example 1

### Synthesis of Antisense Oligonucleotides and Reduction in Levels of MnSOD

All antisense oligonucleotides were synthesized. Phosphorothioate oligodeoxynucleotides (S-oligodeoxynucleotides), in which all phosphodiester linkages were modified, were synthesized, lyophilized, diluted, and stored at −20° C. Oligonucleotides were chosen, purified, and used according to standard procedures Bito et al., Cell, 87, 1203-1214 (1996); Rothstein et al., Neuron, 16, 675-686 (1996). Oligonucleotides were chosen to exhibit minimal self-complementarity according to analysis with the computer program OLIGO 4 (National Biosciences, Plymouth, Minn.). All sequences chosen were specific and unrelated to any other sequence in GenBank.

Antisense oligos were designed for the various antioxidant proteins. In the experimental tests, control cultures or animals received either no oligonucleotide, or sense or random oligonucleotide (in which the base composition and extent of phosphodiester linkages were identical to that of the parent antisense oligo, but the sequence was randomly assigned). Mismatch oligos were used as controls. Thus, controls that were used for MnSOD oligo 2 were:

| | |
|---|---|
| Oligo 2 | |
| 5′ ACA CTG CCC GGC TCA ACA TG | (SEQ ID NO: 2) |
| Sense | |
| 5′ CAT GTT GAG CCG GGC AGT GT | (SEQ ID NO: 8) |
| Mismatch | |
| 5′ ACA CTA CCC AGC TCG ACA TG | (SEQ ID NO: 9) |
| Scrambled | |
| 5′ CTA CAG CCG GCC GTA AAC TC | (SEQ ID NO: 10) |
| Oligo 3 | |
| 5′ CAT GCT GCT AGT GCT GGT GC | (SEQ ID NO: 3) |

Oligos were reconstituted in serum-free medium and filtered before addition to the cultures.

In order to improve penetration of the antisense oligos into certain cancer cells, various delivery vehicles can be used. For example, Lipofectamine was successfully used. Another vehicle that was successfully used with antisense oligos was DOTAP (N-[1-(2,3-dioleoyloxy)propyl]-N,N,N-trimethylammonium methyl sulfate), a cationic diacylglycerol in multilamellar liposomal form. Lin F and Girotti AW., Archives of Biochemistry and Biophysics, 352, 51-58 (1998). Before addition to cells, antisense oligos or control oligos were mixed with DOTAP. The proportion of oligo to DOTAP is typically 0.3:1.0 (w/w). Other vehicles are available commercially.

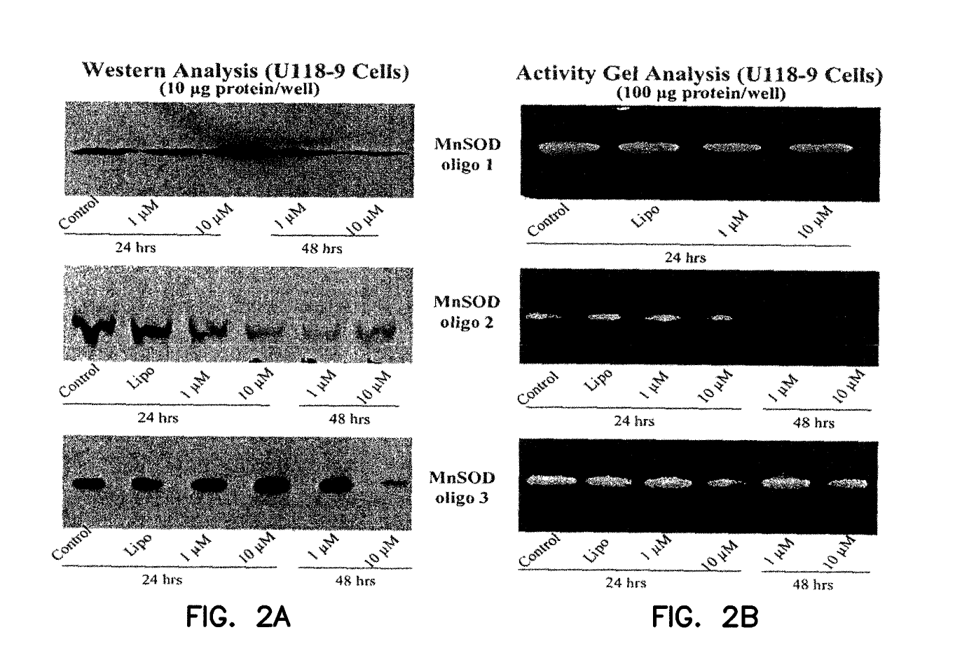

Experiments were then performed with the MnSOD antisense oligos. First it was shown that the oligos inhibited MnSOD immunoreactive protein and activity. When U118-9 human glioma cells were about 50% confluent, they were washed with serum-free media. Then 1 μM oligo and 8 μM Lipofectamine or 10 μM oligos and 16 μM Lipofectamine were added to the cells for 6 hours in serum-free media. After 6 hours, the media was removed and media with serum added to the cells. Cells were harvested at 24 and 48 hours after oligo treatment. The results are shown in FIGS. 2A and 2B. Oligo 2 inhibited both MnSOD immunoreactive protein and MnSOD activity, while oligos 1 and 3 showed no inhibition.

### Example 2

### Modulation of MnSOD Activity in Tumor Cells

The goal in this experiment was to exploit the differences between normal and tumor tissue by modulating manganese superoxide dismutase (MnSOD) activity in tumor cells to achieve tumor-specific cytotoxicity. To date, there is no known specific inhibitor of MnSOD. The strategy was to inhibit MnSOD in human tumor cell lines with an antisense oligodeoxynucleotide (ODN) to the MnSOD transcriptional and translational start sites.

Human breast cancer cells, MCF-7, and human glioma cells, U118-9 were seeded at a density of 40-60% confluency, approximately 150,000 cells for a 6 well dish and 300,000 cells per 60 mm dish in full media. Cells were allowed to attach overnight. For 6-well dishes, a final antisense treatment volume of 1 mL was used to cover the cells. The antisense oligomer and LIPOFECTIN® treatment was prepared. In tube A, a minimal amount of serum-free media and 8 μM LIPOFECTIN® (to be combined with 1 μM oligomer) or 16 μM LIPOFECTIN® (to be combined with 10 μM oligomer) were added to 1.5 ml microfuge tubes to allow for micelle formation at room temperature for 35-45 minutes. In tube B, a minimal amount of serum-free media and 1 or 10 μM antisense MnSOD or catalase was added and incubated for 10-15 minutes. In order for the micelle to incorporate the oligomer, tube A and B was gently mixed together and allowed to incubate at room temperature for 10-15 minutes. The volume was then brought up to 1 mL for 6 well dishes, or the recipe was doubled for 60 mm dishes. The cells were then washed twice with serum-free media and the LIPOFECTIN® plus antisense oligomer mixture was added to the cells for 6 hours at 37° C. After 6 hours the media is changed back to complete media. The cells were scrape harvested at 24 or 48 hours. In order to see the MnSOD antisense effect, 10 nM TNFα was added to induce the MnSOD protein expression when the media was changed. The media was changed after overnight TNFα exposure.

After treatment with antisense human MnSOD, human glioma cells (U118-9) and human breast cancer cells (MCF-7) displayed a 50% decreased MnSOD protein expression and enzyme activity compared to control treatments. When MnSOD was induced in U118-9 cells by exposure to TNFα, cells treated with antisense MnSOD oligodeoxynucleotide showed a two-fold lower induction of MnSOD expression compared to cells treated with the LIPOFECTIN® alone, mismatch, scrambled, and sense oligodeoxynucleotide controls.

MCF-7 xenografts were treated in vivo with antisense MnSOD by intratumoral injection. The results suggested that blocking MnSOD gene expression increased the percentage of tumor-free animals over those treated with LIPOFECTIN® alone, mismatch, scrambled, and sense oligodeoxynucleotide controls. Thus, antisense human MnSOD is effective in blocking the enzymatic function of MnSOD. The antisense oligodeoxynucleotide model is the first to inhibit human MnSOD activity directly and successfully.

### Example 3

### Antisense Oligodeoxynucleotide Manganse Superoxide Dismutase Activity

Antisense oligodeoxynucleotide (ODN) manganese superoxide dismutase (MnSOD) inhibits MnSOD protein expression and cell viability. Antisense ODN MnSOD can also inhibit tumor cell growth and prolong survival of nude mice.

Materials and Methods

Cell culture: Human breast cancer cells, MCF-7, were grown in 90% RPMI and 10% FBS. Human non-tumorigenic epithelial cells, were grown in 90%, 10% FBS. Human Melanoma cells, PS 1273, were grown in 90% RPMI 1640 and 10% FBS. MCF-7 and Melanoma cell lines were grown in 100 units/ml penicillin, 100 μg/ml streptomycin, and 0.25 μg/ml amphotericin B at standard conditions. Cells were seeded at a density of 40-60% confluencey, approximately 500,000 cells per 60 mm dish in full media or 1×10⁶ cells per 100 mm culture dish. Cells were treated as in the oligodeoxynucleotide incorporation methodology. For the clonogenic survival assay, 500 cells were plated per well in a 6 well plate and allowed to attach. Colonies were allowed to grow for 10 days. Cells were fixed and stained in 0.1% crystal violet and 2.1% citric acid.

Western and Activity Gel Analysis: For western analysis, cells were scrape harvested in PBS, pelleted, and sonicated in a minimum amount of PBS. Protein was estimated using Bradford methodology. Ten or 30 μg protein was electrophoresed and assayed for immunoreactivity. The SOD activity gel assay is based on the inhibition of the reduction of nitroblue tetrazolium (NBT) by SOD. MnSOD expression was visualized by the addition of 5 mM NaCN which inhibits CuZnSOD activity.

In vivo experimentation: 2×10⁶ MCF-7 cells were injected subcutaneously into the flank region of female nude mice (Harlan). Tumors were allowed to grow to approximately 70 mm³ (5 mm×5 mm×5mm). 1 mg/kg ODN combined with 8 μM LIPOFECTIN® in serum free EMEM was injected intratumorally every other day for three weeks. Animal survival was noted every two weeks.

When designing an antisense oligodeoxynucleotide (OND), the OND should be at least 11-15 nucleotides long, but no longer than 20-25 nucleotide bases. It should target the initiation codon (AUG/ATG). The phosphodiester bond between nucleotides should be modified to a phosphorothioated backbone for increased stability. G quartets should be avoided as the G residue itself can target hybridization to mRNA. Further, controls should be properly designed regarding mismatch, scrambled, and sense regions.

Oligodeoxynucleotide Incorporation: LIPOFECTIN® TransfectionCells were washed with serum-free media twice. 1 μM oligomer and 8 μM LIPOFECTIN® were added to cells for 6 hours in serum-free media or 10 μM oligomer and 16 μM LIPOFECTIN® were added to cells for 6 hours in serum-free media. After 6 hours the oligo was removed and full serum was added back to the dishes. Cells were harvested 24 or 48 hours post oligomer incorporation.

Effectene Transfection: Cells were plated and allowed to attach overnight in complete media. 1 μM oligomer and 10 μM Effectene were prepared and added to culture dished and allowed to incubate for 24 hours. After 24 hours the oligo was removed and full serum was added back to the dishes. Cells were harvested 48 hours post oligomer incorporation or stained clonogenic survival at day 10 post transfection.

10 μM ODN Incorporation: Cells were plated and allowed to attach overnight in complete media. 10 μM oligomer was added directly into the media and allowed to incubate for 24 hours. After 24 hours the oligo was removed and full serum was added back to the dishes. Cells were harvested 48 hours post oligomer incorporation or stained clonogenic survival at day 10 post transfection.

Results

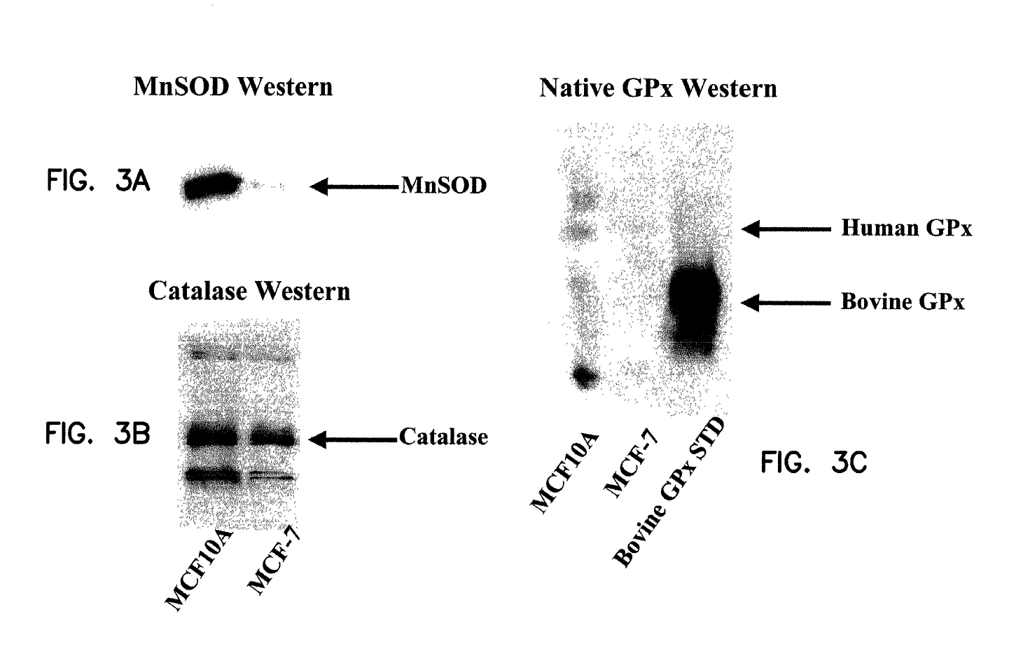

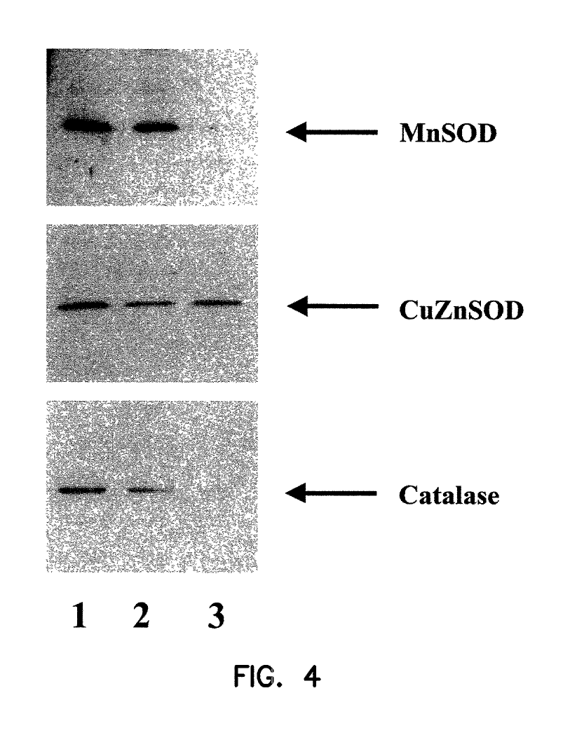

As shown in FIGS. 3A-3C, transformed MCF10A and malignant MCF-7 breast cancer cells differ in antioxidant enzyme expression. MnSOD was high in MCF10A and low in MCF-7, while glutathione peroxidase (GPx) was low in both MCF-7 and MCF10A cell lines. As shown in FIG. 4, antisense MnSOD inhibited MnSOD protein expression at 48 hours in MCF-7 cells. Catalase protein levels also decreased slightly.

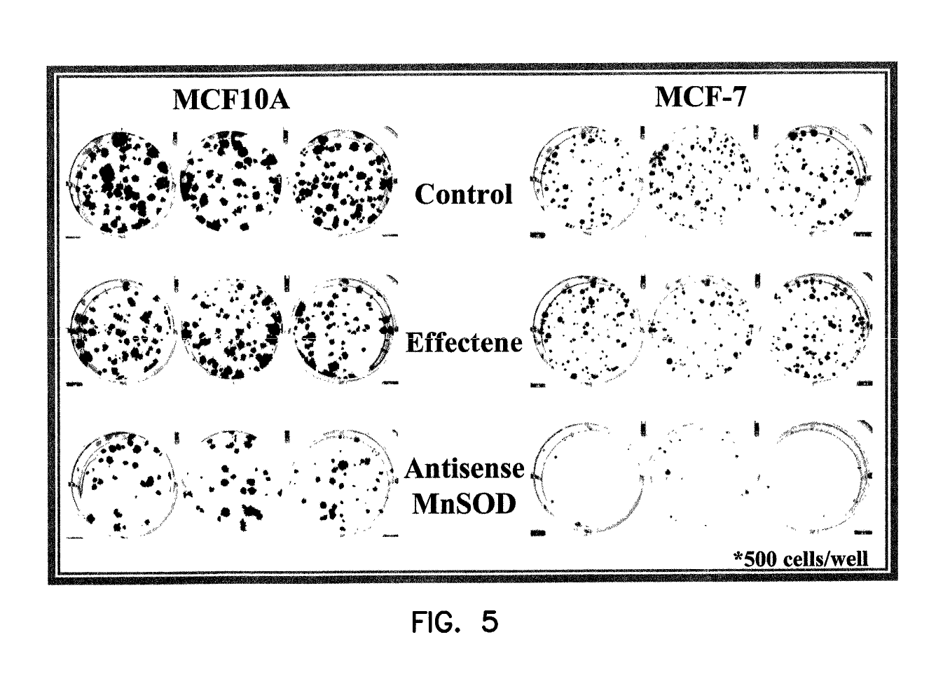

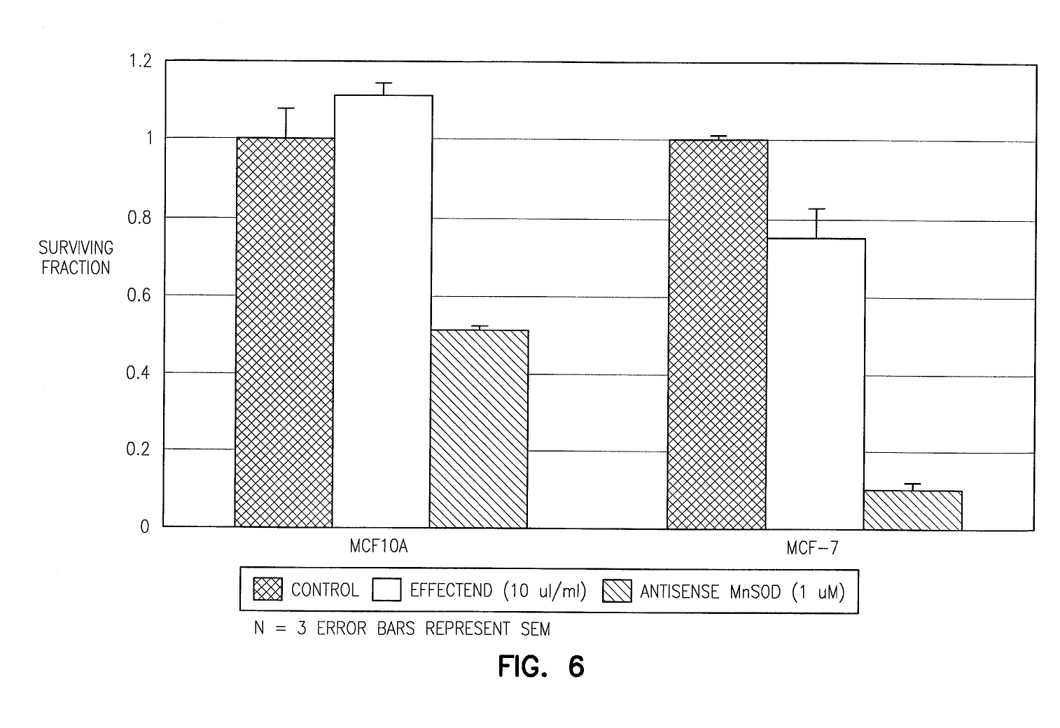

Breast cancer cells have decreased clonogenic survival when treated with 1 μM antisense MnSOD and Effectene (10 μl/ml) for 24 hours, as shown in FIG. 5. MCF-7 cells showed a dramatic loss of colony formation compared to the non-malignant MCF10A cells. Antisense MnSOD differentially inhibited viability of transformed verses malignant breast cancer cells, as shown in FIG. 6. Treatment of MCF10A transformed cells for 24 hours with antisense MnSOD (1 μM) decreased clonogenic survival by 50% while MCF-7 cells a surviving fraction of 10%.

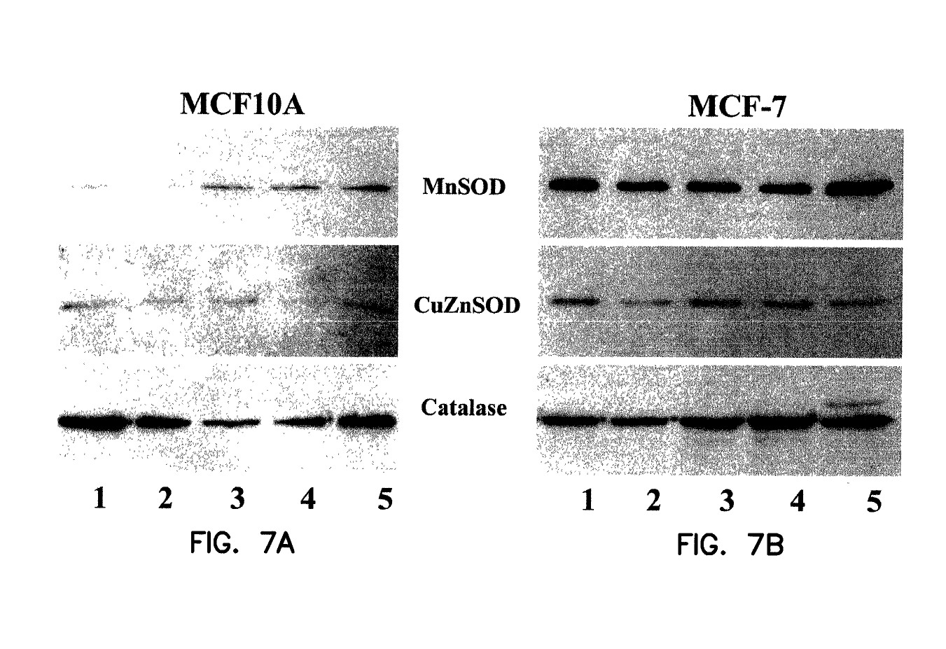

As shown in FIGS. 7A and 7B, antisense ODN successfully inhibited MnSOD in MCF10A cells and MCF-7 cells. Control ODNs have no effect on the MCF10A cells while the scrambled and mismatch oligos may also lower MnSOD protein levels. The ODNs do no effect the other antioxidant enzymes tested. Cells were treated with 10 μM ODN only for 24 hours.

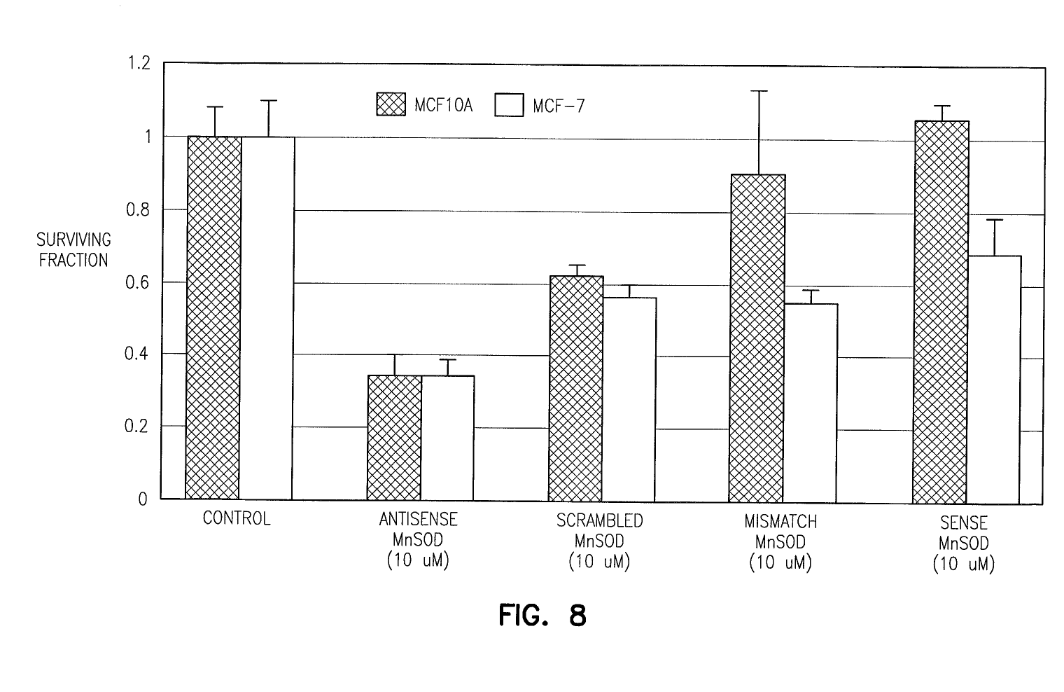

Antisense MnSOD decreased the clonagenic survival of MCF10A and MCF-7 cells 3-fold verses the untreated control cells, as seen in FIG. 8.

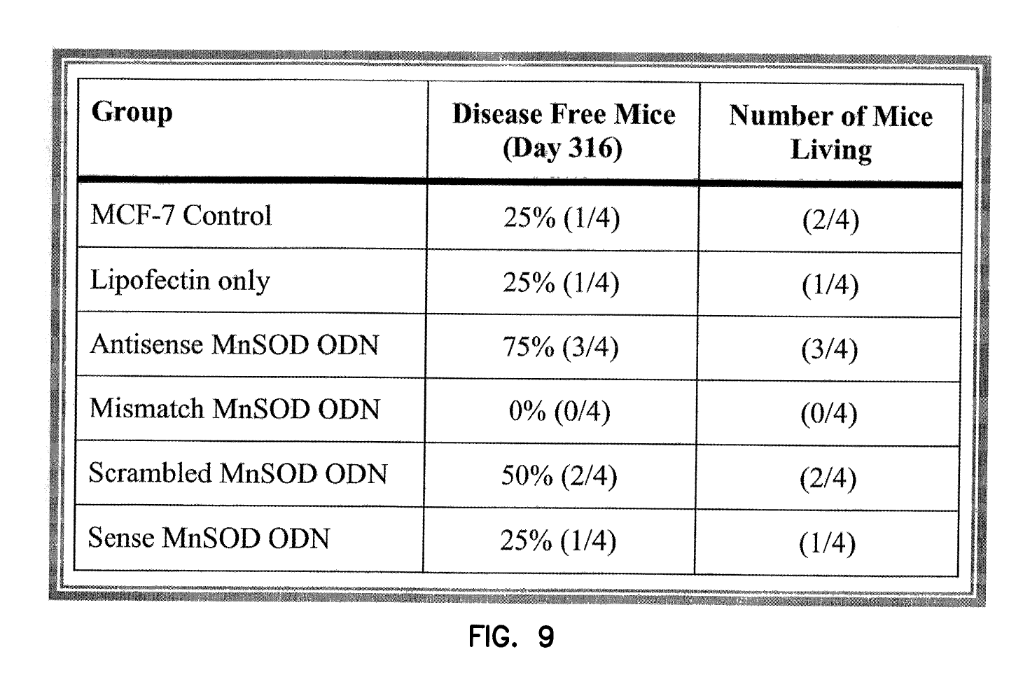

Antisense MnSOD decreased the survival of the two cell lines by half compared to the ODN controls. As seen in FIG. 9 MnSOD oligo 2 increased the number of disease free mice initially bearing MCF-7 tumors compared with control treated tumors at day 316.

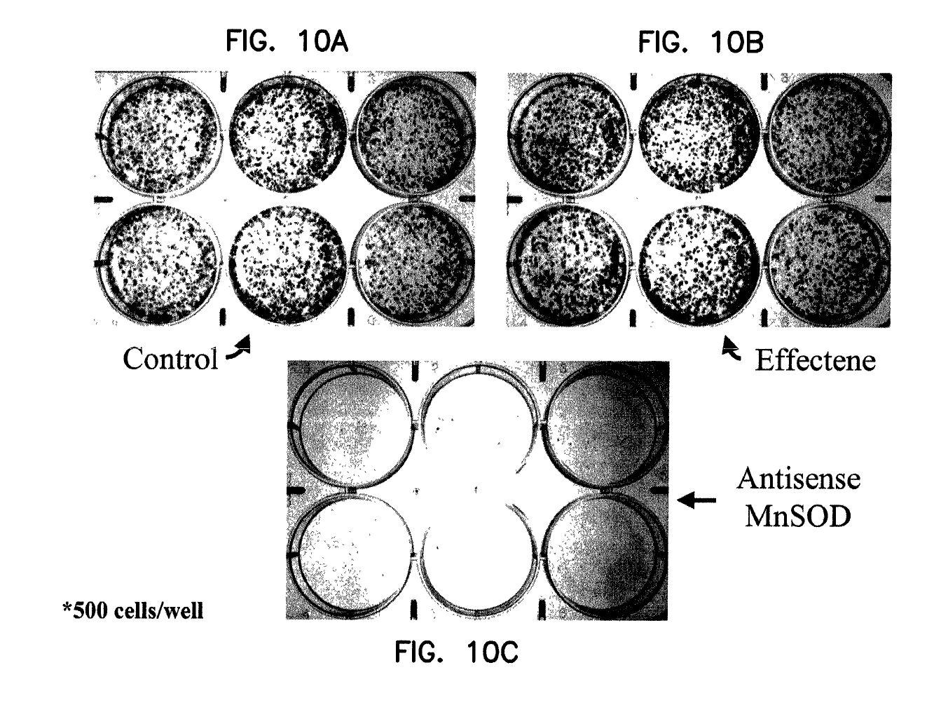

Human melanoma cells treated with 1 μM antisense MnSOD and Effectene (10 μl/ml) have decreased clonogenic survival as seen in the cloning dishes depicted in FIGS. 10A-10C.

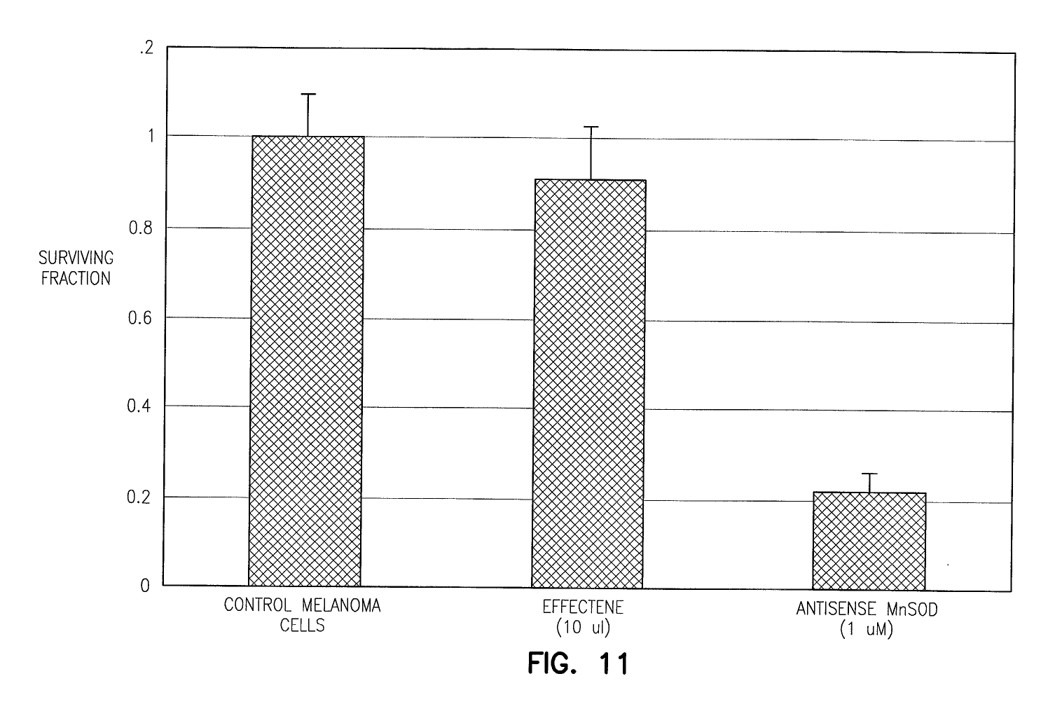

Antisense MnSOD inhibited human melanoma viability when treated with antisense MnSOD (1 μM) for 24 hours, as seen in FIG. 11. The surviving fraction was only 20%, a 5-fold decrease in the clonagenic fraction.

Conclusion

Antisense oligodeoxynucleotide MnSOD effectively decreased the protein expression and clonagenic survival in both MCF10A and MCF-7 cells. The decrease in protein expression of MCF10A was less than that of MCF-7 cells. MCF-7 tumors treated with antisense MnSOD increased the percentage of tumor free animals over those treated with control ODN.

All patents and publications are incorporated by reference herein, as though individually incorporated by reference. Although preferred embodiments of the invention are described herein in detail, it will be understood by those skilled in the art that variations and modifications may be made thereto without departing from the spirit of the invention or the scope of the invention defined by the claims.

### REFERENCES

1. Sun Y, Oberley L W, Oberley T D, Elwell J H, Sierra-Rivera E. Lowered antioxidant enzymes in spontaneously transformed embryonic mouse liver cells in culture, Carcinogenesis, 14, 1457-1463 (1993).

2. Spitz D R, Elwell J H, Sun Y, Oberley L W, Oberley T D, Sullivan S J, Roberts R J. Oxygen toxicity in control and H₂O₂-resistant Chinese hamster fibroblast cell lines, Archives Biochem Biophys, 279, 249-260 (1990).

3. Oberley T D, Oberley L W, Slattery A F, Elwell J H. Immunohistochemical localization of glutathione-S-transferase and glutathione peroxidase in adult Syrian hamster tissues and during kidney development, American J Pathol, 139 355-369 (1991).

4. St. Clair D K, Oberley L W. Manganese superoxide dismutase expression in human cancer cells: a possible role of mRNA processing, Free Rad Res Commun, 12-13, 771-778 (1991).

5. Yan T, Oberley, L W, Zhong W, and St. Clair D K. Manganese-containing superoxide dismutase overexpression causes phenotypic reversion in SV40-transformed human lung fibroblasts, Cancer Res 56, 2864-2871 (1996).

6. Church S L, Grant J W, Ridnour L A, Oberley L W, Swanson P E, Meltzer P S, Trent J M. Increased manganese superoxide dismutase expression suppresses the malignant phenotype of human melanoma cells, Proc Natl Acad Sci USA, 90, 3113-3117 (1993).

7. Zhong W, Oberley L W, Oberley T D, and St. Clair D K. Suppression of the malignant phenotype of human glioma cells by overexpression of manganese superoxide dismutase, Oncogene, 14, 481-490 (1997).

8. Gonzalez-Zulueta M, Ensz L M, Mukina G, Lebovitz R M, Zwacka R M, Engelhardt J F, Oberley L W, Dawson V L, and Dawson T M. Manganese superoxide dismutase protects nNOS neurons from NMDA and nitric oxide mediated neurotoxicity, The Journal of Neuroscience, 18 2040-2055 (1998).

9. Wagener B A, Buettner G R, Oberley L W, and Burns C P. Sensitivity of K562 and HL-60 cells to edelfosine, an ether lipid drug, correlates with production of reactive oxygen species, Cancer Res., 58, 2809-2816 (1998).

10. Wagner B A, Buettner G R, Oberley L W, Darby C J, and Bums C Patrick. Myeloperoxidase is involved in H₂O₂-induced apoptosis of HL-60 human leukemia cells. J Biol Chem, 275, 22461-22469 (2000).

11. Brown M R, Miller Jr. F. J., L i W G, Ellingson A N, Mozena J E, Chatterjee P, Engelhardt J F, Zwacka R A, Oberley L W, Spector A A and Weintraub, N L. Overexpression of human catalase inhibits proliferation and promotes apoptosis in vascular smooth muscle cells, Circulation Research, 85, 524-533 (1999).

12. Yan T, Jiang X, Zhang H J, L i S, and Oberley L W. Use of commercial antibodies for detection of the primary antioxidant enzymes, Free Radical Biology and Med 25, 688-693 (1998).

13. Manna S K, Zhang H J, Yan T, Oberley L W, and Aggarwal B B. Overexpression of manganese superoxide dismutase suppresses tumor necrosis factor-induced apoptosis and activation of nuclear transcription factor-kB and activated protein-1, J Biol Chem, 273 13245-13254 (1998).

14. Zhang H J, Yan T, Oberley T D, and Oberley L W. Comparison of effects of two polymorphic variants of manganese superoxide dismutase on human breast MCF-7 cancer cell phenotype, Cancer Research, 59, 6276-6283 (1999).

15. Ambrosone CB, Freudenheim J L, Thompson P A, Bowman E, Vena J E, Marshall J R, Graham S, Laughlin R, Nemoto T, Shields P G. Manganese Superoxide Dismutase (MnSOD) Genetic Polymorphisms, Dietary Antioxidants, and Risk of Breast Cancer, Cancer Res, 59, 602-606 (1999).

16. Li S, Yan T, Yang J-Q, Oberley T D, and Oberley L W. The role of cellular glutathione peroxidase redox regulation in the suppression of tumor cell growth by manganese superoxide dismutase. Cancer Research, 60, 3927-3939 (2000).

17. Oberley L W, Spitz D R. Nitroblue tetrazolium. In: Handbook of Methods for Oxy Radical Research, CRC Press, Boca Raton, 217-220 (1985).

18. Paglia P E, and Valentine W N. Studies on the quantitation and qualitative characterization of erythrocyte glutathione peroxidase, J Lab Clin Med, 70, 158-169 (1967).

19. Beers J r R F, and Sizer I W. A spectrophotometric method for measuring the breakdown of hydrogen peroxidase by catalase, J Biol Chem, 195 133-140 (1952).

20. Ray L E, and Prescott J M. Isolation and some characteristics of glutathione reductase from rabbit erythrocytes, Proc Soc Exp Biol (NY), 148, 402-409 (1975).

21. Wong GHW, Elwell J H, Oberley L W, and Goeddel D V. Manganous superoxide dismutase is essential for cellular resistance to cytotoxicity of tumor necrosis factor, Cell 58, 923-931 (1989).

22. David B J. Disc electrophoresis-II. Method and application to human serum proteins, Ann NY Acad Sci 121, 404-427 (1964).

23. Beauchamp C, Fridovich I. Superoxide dismutase: improved assays and an assay applicable to acrylamide gels, Anal Biochem, 44, 276-287 (1971).

24. Akerboom TPM, Sies T. Assay of glutathione, glutathione disulfide and glutathione mixed disulfides in biological samples. In: Methods of Enzymology, vol 77, Jakoby W, ed, Academic Press, New York, 373-382 (1981).

25. Bozeman P M, Learn D B et al. Assay of the human leukocyte enzymes myeloperoxidase and eosinophil peroxidase, J Immunol Methods, 126, 125-133 (1990).

27. McCormick, M L, Buettner G R, and Britigan B E. The spin trap α-(4-pyridyl-1-oxide)-N-tert-butylnitrone stimulates peroxidase-mediated oxidation of deferoxamine, J Biol Chem, 270, 29265-29269 (1995).

28. Lowry O H, Rosebrough N H, Farr A L, Randall R J. Protein measurement with the Folin phenol reagent, J Biol Chem, 193, 265-275 (1951).

29. Burton K. A study of the conditions and mechanisms of the diphenylamine reaction for the colorimetric estimation of deoxyribonucleic acid, Biochem J, 62, 215-223 (1956).

30. Oberley L W, McCormick M L, Sierra-Rivera E, Kasemset St. Clair D. Manganese superoxide dismutase in normal and transformed human embryonic lung fibroblasts, Free Rad Biol Med, 379-384 (1989).

31. Ho Y-S, Crapo J. Isolation and characterization of complimentary DNA's encoding human MnSOD, FEBS Letters, 229, 256-261 (1988).

32. St. Clair D K, Holland J C. Complimentary DNA encoding human colon cancer manganese superoxide dismutase and the expression of its gene in human cells, Cancer Res. 51, 939-943 (1991).

33. Maniatis T, Fritsch E F, Sanbrooks J. In: Molecular Cloning: A Laboratory Manual, Cold Spring Harbor Laboratory, Cold Springs Harbor, New York (1982).

34. Chirgwin J M, Pozybyla A E, MacDonald R J, Rutter W J. Isolation of biologically active ribonucleic acid from sources enriched in ribonuclease, Biochem, 18,2594-2599 (1979).

35. Aviv H, Leder P. Purification of biologically active globin messenger RNA by chromatography on oligothymidylic acid-cellulose, Proc Natl Acad Sci USA, 69 1408-1412 (1972).

36. Thomas P S. Hybridization of denatured RNA and small DNA fragments transferred to nitrocellulose, Proc Natl Acad Sci USA, 77, 5201-5205 (1980).

37. Davis L G, Dibner M D, Battey J F. In: Basic Methods in Molecular Biology, Elsevier, New York, 44-46, 1986.

38. Southern C G. Detection of specific sequences among DNA fragments separated by gel electrophoresis, Mol Biol, 98, 503-517 (1975).

39. St. Clair D K, Oberley T D, Ho Y S. Overproduction of human Mn-superoxide dismutase modulates paraquat-mediated toxicity in mammalian cells, FEBS Letters, 293, 199-203 (1991).

40. Zwacka R M, Zhou W, Zhang Y, Darby C J, Dudus L, Halldorson J, Oberley L W, and Engelhardt J F. Redox gene therapy for ischemia/reperfusion injury of the liver reduces AP1 and NF-kB activation, Nature Medicine, 4, 698-704 (1998).

41. Lam EWN, Zwacka R, Engelhardt J F, Davidson B L, Domann F E, Yan T, and Oberley L W. Adenovirus-mediated manganese superoxide dismutase gene transfer to hamster cheek pouch carcinoma cells, Cancer Res, 57, 5550-5556 (1997).

42. Elroy-Stein O, Bernstein Y, Groner Y. Overproduction of human Cu/Zn-superoxide dismutase in transfected cells: extenuation of paraquat-mediated toxicity and enhancement of lipid peroxidation, EMBO J, 5, 615-622 (1986).

43. Elroy-Stein O, Groner Y. Impaired neurotransmitter uptake in PC12 cells overexpressing human Cu/Zn-superoxide dismutase-implication for gene dosage effects in Down's syndrome, Cell, 52, 259-267 (1988).

44. Sierra-Rivera E. Multistep nature of neoplastic transformation in rat embryo fibroblasts, Ph.D. thesis, The University of Iowa, December, 1987.

45. Ausubel F M, Brent R, Kingston R E, Moore D D, Seidman J G, Smith J A, Struhl K S. In: Current Protocols in Molecular Biology, John Wiley and Sons, New York, Chapter 9, 1987.

46. Bito H, Deisseroth K et al. CREB phosphorylation and dephosphorylation: a Ca(2+)- and stimulus-duration dependent switch for hippocampal gene expression, Cell 87,1203-1214 (1996).

47. Rothstein J D, Dykes-Hoberg M et al. Knockout of glutamate transporter reveals a major role for astroglial transport in excitotoxicity and clearance of glutamate, Neuron, 16, 675-686 (1996).

48. Lin F and Girotti A W. Hemin-enhanced resistance of human leukemia cells to oxidative killing: antisense determination of ferritin involvement, Archives of Biochemistry and Biophysics, 352, 51-58 (1998).

What is claimed is:

1. An oligonucleotide comprising an antisense nucleic acid sequence that specifically binds to an antioxidant enzyme start codon, wherein the sequence is about 18 to 26 nucleotides in length.

2. The oligonucleotide of claim 1, wherein the nucleic acid is about 20 nucleotides in length.

3. The oligonucleotide of claim 1, wherein the nucleic acid sequence is phosphothiolated.

4. The oligonucleotide of claim 1, wherein the antioxidant enzyme is manganese superoxide dismutase, copper and zinc superoxide dismutase, catalase, phospholipid glutathione peroxidase, or cytosolic glutathione peroxidase.

5. The oligonucleotide of claim 4, wherein the antioxidant enzyme is manganese superoxide dismutase, catalase, or phospholipid glutathione peroxidase.

6. The oligonucleotide of claim 1, wherein the nucleic acid sequence is 90% identical to the nucleic acid encoding an antioxidant enzyme.

7. The oligonucleotide of claim 1, wherein the nucleic acid sequence is 100% identical to the nucleic acid encoding an antioxidant enzyme.

8. A method of treating an antioxidant enzyme malfunction disorder in a mammal comprising reducing antioxidant enzyme levels in a cell by administering a therapeutic agent comprising an oligonucleotide of claim 1.

9. The method of claim 8, wherein the disorder is a tumor, heart disease, arthritis, or neurodegenerative disease.

10. The method of claim 9, wherein the disorder is a tumor.

11. The method of claim 9, wherein the therapeutic agent is injected into the tumor.

12. The method of claim 8, wherein the mammal is a human.

13. The method of claim 8, wherein the therapeutic agent further comprises a delivery vehicle.

14. The method of claim 13, wherein the delivery vehicle is lipofectamine or N-[1-(2,3-dioleoyloxy)propyl]-N,N,N-trimethylammonium methyl sulfate (“DOTAP”).

15. The method of claim 8, wherein the nucleic acid sequence is phosphothiolated.

16. The method of claim 8, wherein the antioxidant enzyme is manganese superoxide dismutase, copper and zinc superoxide dismutase, catalase, phospholipid glutathione peroxidase, or cytosolic glutathione peroxidase.

17. The method of claim 16, wherein the antioxidant enzyme is manganese superoxide dismutase, catalase, or phospholipid glutathione peroxidase.

18. The method of claim 8, wherein the nucleic acid sequence is 90% identical to the nucleic acid encoding an antioxidant enzyme.

19. The method of claim 8, wherein the nucleic acid sequence is 100% identical to the nucleic acid encoding an antioxidant enzyme.


SEQUENCE LISTING (12 sequences)

SEQ ID NO: 1 - DNA, 20 bp, Homo sapiens
ccggctcaacatgctgctag

SEQ ID NO: 2 - DNA, 20 bp, Homo sapiens
acactgcccggctcaacatg

SEQ ID NO: 3 - DNA, 20 bp, Homo sapiens
catgctgctagtgctggtgc

SEQ ID NO: 4 - DNA, 20 bp, Homo sapiens
ggatcccggctgtcagccat

SEQ ID NO: 5 - DNA, 20 bp, Homo sapiens
catagcgtgcggtttgctct

SEQ ID NO: 6 - DNA, 20 bp, Homo sapiens
gccgaggctcatcgcggcgg

SEQ ID NO: 7 - DNA, 20 bp, Homo sapiens
caaaggcggccgaggctcat

SEQ ID NO: 8 - DNA, 20 bp, Homo sapiens
catgttgagccgggcagtgt

SEQ ID NO: 9 - DNA, 20 bp, Artificial Sequence
  An artificial oligonucleotide
acactacccagctcgacatg

SEQ ID NO: 10 - DNA, 20 bp, Artificial Sequence
  An artificial oligonucleotide
ctacagccggccgtaaactc

SEQ ID NO: 11 - DNA, 325 bp, Homo sapiens
gcagatcggcggcatcagcggtagcaccagcactagcagcatgttgagccgggcagtgtgcggcaccagcaggcagctggctccggttttggggtatctgggctccaggcagaagcacagcctccccgacctgccctacgactacggcgccctggaacctcacatcaacgcgcagatcatgcagctgcaccacagcaagcaccacgcggcctacgtgaacaacctgaacgtcaccgaggagaagtaccaggaggcgttggccaagggagatgttacagcccagatagctcttcagcctgcagtgaagttcaatggtggtggtcat

SEQ ID NO: 12 - protein, 95 aa, Homo sapiens
MLSRAVCGTSRQLAPVLGYLGSRQKHSLPDLPYDYGALEPHINAQIMQLHHSKHHAAYVNNLNVTEEKYQEALAKGDVTAQIALQPALKFNGGGH